(19) United States
(12) Patent Application Publication
(10) Pub. No.: US 2009/0209848 A1
(43) Pub. Date: Aug. 20, 2009

(54) GEL MATERIAL FOR USE IN MRI

(75) Inventors: John K. Saunders, Winnipeg (CA); Stephen G. Hushek, Wauwatosa, WI (US); Victoria D.M. Hornblower, Winnipeg (CA)

Correspondence Address: ADE & COMPANY INC., 2157 Henderson Highway, WINNIPEG, MB, R2G1P9, CA

(73) Assignee: IMRIS INC.,, Winnipeg (CA)

(21) Appl. No.: 12/034,002

(22) Filed: Feb. 20, 2008

(51) Int. Cl. A61B 5/055

(52) U.S. Cl. 600/422

(57) ABSTRACT

In MR imaging of a body part of a patient in an intra-operative procedure, the body part is supported using gel pads where the gel material is arranged such that the presence of the gel material does not generate an image in the analysis of the RF signal from the location so that the pad is invisible in the MR image. This can be obtained by using a water based gel material and providing an additive such as manganese chloride to reduce the relaxation time of the water molecules to a value which is sufficiently short that the molecules relax before the acquisition of the signals by the RF receive coil. This can be achieved by using a non-water based gel or a heavy water based gel.

This invention relates to a gel material for use in patient positioning devices in magnetic resonance imaging and particularly for use in extended procedures where MRI is carried out while the patient remains on the head support.

### BACKGROUND OF THE INVENTION

In order to support a patient's head and/or body and to maintain the head comfortably during a surgical procedure which may last for 12 hours or longer there are many designs of positioning aids which have been proposed. In many cases a pad is used to actually make engagement with the head or body part and in order to make the pads comfortable commonly a liquid gel is employed as a filler for the pad.

One example is disclosed in U.S. Pat. No. 7,117,551 (Dinkler) issued Oct. 10, 2006 entitled “Head support System”. This discloses a head support system for use with a patient support where the head support system has a support structure with a crossbar extending transverse to, and connected to, the patient support. The head support device is removably mountable to a portion of the support structure exclusive of the crossbar and provides a static support for the patient's head. A head stabilization device is removably mountable to the crossbar of the support structure and stabilizes the patient's head. Pads engaging the head include a gel type filler material.

Such supports are therefore commonly used in surgery. One difficulty arises where the patient is also to be imaged on an intra-operative basis using an MRI system for example using the medical procedure disclosed in U.S. Pat. No. 5,735,278 (Hoult et al) issued Apr. 7, 1998 where a magnet is movable relative to a patient and relative to other components of the system. The moving magnet system allows intra-operative MRI imaging to occur more easily in neurosurgery patients, and has additional applications for liver, breast, spine and cardiac surgery patients. The disclosure of this patent is incorporated herein by reference.

### SUMMARY OF THE INVENTION

It is one object of the invention to provide an improved patient anatomy support which can be used in an MRI intra-operative procedure.

According to one aspect of the invention there is provided a method for imaging of a body part of a patient comprising:

providing a location for a patient mounted in a static magnetic field, providing adjacent to the location a transmit coil for generating a radio-frequency magnetic field,

providing adjacent to the location of the sample a receive coil for receiving an RF signal from material in the location generated in response to the transmitted radio frequency magnetic field and the static magnetic field;

analyzing the RF signal to generate an image from the signal;

at the location supporting the body part of the patient by engaging the body part with one or more pads each formed by an outer flexible envelope containing a gel material;

and arranging the gel material such that the presence of the gel material in the location does not generate an image in the analysis of the RF signal from the location so that the pad is invisible in the image of the body part of the patient.

According to a second aspect of the invention there is provided a head support for use in a surgical procedure in effecting imaging of the head of a patient on an intra-operative basis at a location using magnetic resonance imaging, where an image is generated from an RF signal obtained from the location, the head support comprising:

a mechanical structure for attachment to a support member;

one or more pads carried on the mechanical structure for engaging the head of the patient;

each pad being formed by an outer flexible envelope containing a gel material carried on the mechanical structure;

the gel material being selected and arranged such that the presence of the gel material in the location does not generate an image in the analysis of the RF signal from the location so that the pad is invisible in the image of the body part of the patient.

According to a third aspect of the invention there is provided an anatomic support for use in a surgical procedure in effecting imaging of a part of a patient on an intra-operative basis at a location using magnetic resonance imaging, where an image is generated from an RF signal obtained from the location, the anatomic support comprising:

one or more pads for engaging the part of the patient;

each pad being formed by an outer flexible envelope containing a gel material carried on the mechanical structure;

the gel material being selected and arranged such that the presence of the gel material in the location does not generate an image in the analysis of the RF signal from the location so that the pad is invisible in the image of the body part of the patient.

### DETAILED DESCRIPTION

One example of a mechanical support structure for mounting the head engagement pads is disclosed in U.S. Pat. No. 7,117,551 (Dinkler) issued Oct. 10, 2006 entitled “Head support System”. The disclosure of this patent is incorporated by reference and can be used to show how such pads are constructed and mounted.

The present arrangement relates to the selection and arrangement of the gel material which is used in the pads of for example the above patent so that no illustration of the support itself is necessary herein.

It is in the realm of intra operative MRI that MRI invisible pads will be needed and this will be particularly true for pediatric patients.

Typical gel pads are water based resulting in strong MRI signals during imaging. The objective of this work is to retain all the comfort of the gel pad but eliminate all trace of the liquid material from the MR Image.

One technique method to eliminate the signal from the MR Image in a water based gel pad is to add manganese chloride to the aqueous solution in sufficient concentration to make the relaxation time T2 so short that all signals from the gel pad have de-phased prior to acquisition of the region of interest k space data. The concentration of MnCl₂ will be about 1.5 milli-molar.

T2 is one of the time constants involved in relaxation processes that establish equilibrium following RF excitation. Thus as the high-energy nuclei de-phase and realign they transfer energy at rates which are recorded to provide information about the material they are in. The realignment of nuclear spins with the static magnetic field is termed longitudinal relaxation and the time required for a certain percentage of the tissue's nuclei to realign is termed T1, which is typically about 2-5 seconds in human tissue. T2-weighted imaging relies upon de-phasing of spins following the application of the transverse radio-frequency pulse; the transverse relaxation time is termed T2, which is typically less than 100 ms for tissue.

The use of MnCl₂ to reduce the relaxation times of water hydrogen atoms is known and this compound is used as a contrast agent because of its ability to reduce the T1 and T2 of water hydrogen atoms. Other materials having the same effect exist and examples are CoCl₂, CrCl₃ etc. It is known that the majority of the ionic compounds of the transition metals including copper, iron, manganese, chromium, cobalt etc reduce both T1 and T2 when dissolved in aqueous media. The most important criterion is that there are unpaired electrons. Many of these metals exist in different ionic states such as for iron, ferrous (FeCl₂ for example) and ferric (FeCl₃). The majority of these compounds, when dissolved in water, cause rapid relaxation of the water hydrogen nuclei.

The choice of manganese is desirable but not essential because it is probably the safest of the above compounds. For example it is known that it can be injected into animals as a contrast agent at concentrations higher than provided here and as defined above.

An alternative method of eliminating all proton signals from the gel is to prepare the gel by use of a non-water based gel material as the liquid rather than to use water, such as a hydrocarbon and of which one particular example is poly fluorinated hydrocarbon. The poly fluorinated hydrocarbon is preferably selected so as to be safe for human use. Thus for example poly fluorinated blood emulsions, which are used as blood substitutes, can be used as the basis for the gel. However, other non-water based gel materials such as carbon tetrachloride can be used, provided they do not contain hydrogen atoms.

Another alternative is to replace the normal water in a gel material with deuterium oxide (heavy water) which does not have any proton signals and therefore is not observed in a standard image.

The advantage of the invention is that MR images of the subject of interest can be obtained without any interference from the gel pads.

The novelty arises from the application of a doping technique commonly known in the MR industry to a surgical positioning device.

The use of poly fluorinated hydrocarbons or deuterium oxide in gels is novel.

The MRI invisible gel head supports described are very useful since signal from the gel pads can impact the MR image in several different ways and not allow an ideal image of the patient's head.

Since various modifications can be made in my invention as herein above described, and many apparently widely different embodiments of same made within the spirit and scope of the claims without department from such spirit and scope, it is intended that all matter contained in the accompanying specification shall be interpreted as illustrative only and not in a limiting sense.

1. A method for imaging of a body part of a patient comprising:
providing a location for a patient mounted in a static magnetic field,
providing adjacent to the location a transmit coil for generating a radio-frequency magnetic field,
providing adjacent to the location a receive coil for receiving an RF signal from material in the location generated in response to the transmitted radio frequency magnetic field and the static magnetic field;
analyzing the REF signal to generate an image from the signal;
at the location supporting the body part of the patient by engaging the body part with one or more pads each formed by an outer flexible envelope containing a gel material;
and arranging the gel material such that the presence of the gel material in the location does not generate an image in the analysis of the RF signal from the location so that the pad is invisible in the image of the body part of the patient.

2. The method according to claim 1 wherein the gel material is formed from a water based gel material and wherein an additive is included which acts to reduce the relaxation time of the water molecules to a value which is sufficiently short that the molecules relax before the acquisition of the signals by the RE receive coil which are used in the generation of the image.

3. The method according to claim 1 wherein the gel material is formed from a water based gel material and wherein manganese chloride is added to the gel material.

4. The method according to claim 3 wherein the concentration of MnCl₂ is about 1.5 milli-molar.

5. The method according to claim 1 wherein the additive is added to the aqueous solution in sufficient concentration to make T2 so short that all signals from the gel pad have relaxed prior to acquisition of the region of interest k space data.

6. The method according to claim 1 wherein all proton signals are eliminated from the gel by forming the gel from a non-water based material.

7. The method according to claim 6 wherein the gel is a hydrocarbon based gel.

8. The method according to claim 7 wherein the gel is a poly fluorinated hydrocarbon.

9. The method according to claim 8 wherein the poly fluorinated hydrocarbon is selected so as to be safe for human use.

10. The method according to claim 1 wherein the gel material is formed from a deuterium oxide water based gel material.

11. The method according to claim 1 wherein the imaging is carried out on an intra-operative basis during an operating procedure on the patient.

12. A head support for use in a surgical procedure in effecting imaging of the head of a patient on an intra-operative basis at a location using magnetic resonance imaging, where an image is generated from an RF signal obtained from the location, the head support comprising:
a mechanical structure for attachment to a support member;
one or more pads carried on the mechanical structure for engaging the head of the patient;
each pad being formed by an outer flexible envelope containing a gel material carried on the mechanical structure;
the gel material being selected and arranged such that the presence of the gel material in the location does not generate an image in the analysis of the RF signal from the location so that the pad is invisible in the image of the body part of the patient.

13. An anatomic support for use in a surgical procedure in effecting imaging of a part of a patient on an intraoperative basis at a location using magnetic resonance imaging, where an image is generated from an RF signal obtained from the location, the anatomic support comprising:
one or more pads for engaging the part of the patient;
each pad being formed by an outer flexible envelope containing a gel material carried on the mechanical structure;
the gel material being selected and arranged such that the presence of the gel material in the location does not generate an image in the analysis of the RF signal from the location so that the pad is invisible in the image of the body part of the patient.
